(19) United States
(12) Patent Application Publication
(10) Pub. No.: US 2025/0186129 A1
(43) Pub. Date: Jun. 12, 2025

(54) TRACKING SYSTEM AND METHOD OF TRACKING REAL-TIME POSITIONS BY A MOVEABLE SENSOR WITHIN A PATIENT'S BODY WITH A TRACKING SYSTEM

(71) Applicant: ABIOMED Europe GmbH, Aachen (DE)

(72) Inventors: Katrin Lunze, Aachen (DE); Paul Zech, Aachen (DE)

(21) Appl. No.: 18/846,008

(22) PCT Filed: Mar. 15, 2023
(86) PCT No.: PCT/EP2023/056544

§ 371 Date: Sep. 11, 2024

(30) Foreign Application Priority Data
Mar. 15, 2022 (EP) 22162194.9

(51) Int. Cl. A61B 34/20

(52) U.S. Cl. CPC A61B 34/20; A61B 2034/2051

(57) ABSTRACT

The present invention relates to a tracking system (10). The tracking system (10) comprises an emitting device (12) configured to establish a measurement volume within at least a part of a patients body, a moveable sensor (16) which is moveable within the measurement volume, a reference sensor (18) establishing a first coordinate system within the measurement volume, a storage device (22) comprising at least one virtual anatomical model VM of at least a part of the patients body B, wherein the at least one virtual anatomical model has a second coordinate system, and a controller (20). The controller (20) is configured to align the first coordinate system and the second coordinate system and to translate real-time positions of the moveable sensor (16) in the first coordinate system into positions in the second coordinate system.

### BACKGROUND

The present invention relates to a tracking system comprising an emitting device, a moveable senor, a reference sensor and a controller. The present invention further relates to a method of tracking real-time positions of a moveable sensor within a patient's body with a tracking system. In particular, the tracking system is to be used when introducing a blood pump or a guidewire for a blood pump into a patient's body. The blood pump may be a catheter pump, in particular an intravascular blood pump, an intracardiac blood pump or any other kind of ventricular assist device.

Such blood pumps of different types are known from the prior art and are intended to support the function of a patient's heart, either in a short-term application, in which an intravascular blood pump is placed in the patient for a couple of days or weeks, or in a long-term application, in which the intravascular blood pump is placed in the patient for a couple of weeks or months. The blood pumps may e.g. be inserted into a patient's body through the aorta by using a catheter, or may be placed in the thoracic cavity. During planned surgery, intravascular blood pumps are often introduced into the patient's body using a guidewire, serving as a track for the intravascular blood pump. For example, guidewires and intravascular blood pumps are introduced into the patient's body using fluoroscopy to warrant a correct positioning of the intravascular blood pump in the patient's left ventricle. Commonly, X-ray is used for fluoroscopy.

However, there are certain disadvantages associated with fluoroscopy and, in particular, with X-ray. During planned surgery for placing the blood pump or the guidewire respectively X-ray is not available without restrictions. In addition, the patient and the physician are exposed to a high amount of radiation when conducting X-ray. When it comes to an emergency condition making it necessary to immediately introduce an intravascular blood pump into the patient's body, X-ray is often not available, e.g. at an Immediate Care Unit or in an ambulance car. Furthermore, during emergency conditions intravascular blood pumps are introduced into the patient's body without a guidewire, e.g. directly through a femoral access or an axillary access.

Therefore, the need exists to provide an ameliorated solution for tracking a position of a blood pump or a guidewire for a blood pump when introducing the blood pump or guidewire into a patient's body.

According to a first aspect, a tracking system comprises an emitting device configured to establish a measurement volume within at least a part of a patient's body, a moveable sensor which is moveable within the measurement volume, a reference sensor establishing a first coordinate system within the measurement volume, a storage device comprising at least one virtual anatomical model of at least a part of the patient's body, wherein the at least one virtual anatomical model has a second coordinate system, and a controller being configured to align the first coordinate system and the second coordinate system and to translate real-time positions of the moveable sensor in the first coordinate system into positions in the second coordinate system. The controller may be configured to detect real-time positions of the movable sensor within the first coordinate system.

The virtual anatomical model covers that part of the patient's body which is of interest, e.g. the torso with parts of the legs covering the femoral artery. The measurement volume also covers said part of the patient's body. With the tracking system, the virtual anatomical model of said part of the patient's body can be aligned with the real anatomy, so that the real anatomy of the patient matches the virtual anatomical model. When introducing e.g. an intravascular blood pump through a femoral access via the femoral artery, the movement of the movable sensor can thus be visualized within the virtual anatomical model, which is in alignment with the real anatomy of the patient. This allows for correct placement of the intravascular blood pump without fluoroscopy. In addition, aligning the first coordinate system of the reference sensor and the second coordinate system of the virtual anatomical model allows to detect real-time positions of the movable sensor within the virtual anatomical model independently of the relative movement between the patient and the emitting device.

The controller may be configured to detect real-time positions of the movable sensor within the first coordinate system.

The controller may be configured to align the first coordinate system and the second coordinate system based on at least a detected first position of the moveable sensor within the first coordinate system. Preferably, the detected first position is an average of a plurality of spaced positions of the movable sensor, preferably of five consecutive positions. As the physician introduces the movable sensor together with e.g. the intravascular blood pump e.g. through a femoral access and pushes the same to the aorta, the actual position of the movable sensor (e.g. within the descending aorta) is known and can be used to align the first coordinate system and the second coordinate system.

The controller may be configured to align the first coordinate system and the second coordinate system based on the detected first position and a movement direction of the movable sensor, wherein the movement direction is preferably a linear vector. The movement direction may be calculated from a vector connecting the detected first position and a detected second position, wherein the detected second position is detected after the detected first position, i.e. when the movable sensor has been further introduced into the patient's body. Preferably, the detected second position is an average of a plurality of spaced positions, preferably of five consecutive positions. Further, it is preferable that the detected first position and the detected second position are sufficiently spaced from each other, in particular by at least 2 cm, but preferably by no more than 10 cm. Hence, considering the movement direction further ameliorates the alignment of the first coordinate system and the second coordinate system.

The virtual anatomical model may comprise an identifiable structure and the controller may further be configured to move the identifiable structure together with the second coordinate system to the detected first position. The identifiable structure may be a model of an aortic arch. The identifiable structure preferably is a consistent structure independent of anatomical differences and properties between individual patients. Using an identifiable structure common for all patients further ameliorates the alignment between the first coordinate system and the second coordinate system.

The reference sensor may be a six degrees of freedom sensor. This may allow for a particular accurate alignment of the first coordinate system and the second coordinate system.

The tracking system may further comprise a medical device, wherein the moveable sensor may be attached to the medial device. Preferably, the medical device is catheter pump, like an intravascular blood pump, an intracardiac blood pump or a guidewire for an intravascular blood pump or an intracardiac blood pump. Hence, the real-time positions of the medical device within the patient's body can be tracked while introducing the same. As such, the correct position of the medical device e.g. within the patient's heart can be warranted.

The emitting device may be an electromagnetic field generator. Preferably, the electromagnetic field generator emits a low-intensity, varying electromagnetic field inducing small currents within the movable sensor and the reference sensor. The currents are communicated to the controller and the controller may be further configured to amplify and digitalize the currents to generate digital signals for further computing and processing.

The moveable sensor may be an embedded electromagnetic sensor. The movable sensor may be a five degrees of freedom sensor or a six degrees of freedom sensor. Depending on the needs, a respective movable sensor may be used. For instance, in case the moveable sensor is attached to an intravascular blood pump having a pigtail, the sensor may be a six degrees of freedom sensor so that the relative position of the tip of the pigtail relative to the movement direction of the intravascular blood pump (i.e. along the X-axis) may also be tracked.

The tracking system may further comprise a display device. The display device may be configured to display the real-time positions of the moveable sensor in the second coordinate system of the virtual anatomical model. The display device may be configured to display the real-time positions of the moveable sensor in the virtual anatomical model. This visualizes the real-time positions of the movable sensor to the physician within the virtual anatomical model suitable for the patient's body. In other words, an image of the patient's body with the real-time position of the movable sensor is shown to the physician.

The storage device may further comprise a plurality of selectable virtual anatomical models. The virtual anatomical model to be used may be selected by the physician before introducing the movable sensor into the patient's body. The virtual anatomical model to be used may also be automatically selected by the controller based on input parameters related to the patient. In particular, the input parameters may be sex, age, body height and/or body weight.

The tracking system may further comprise an input device. The input device may be configured to communicate with the controller. The input device may be a touch screen, a keyboard, a cell phone, a wireless input device, a wired input device, a terminal, a tablet, and/or a remote control. The controller may comprise the storage device. The storage device may be a non-volatile storage device.

According to a second aspect, a method of tracking real-time positions of a moveable sensor within a patient's body with a tracking system, in particular with a tracking system according to the first aspect, comprises the following steps: providing an emitting device establishing a measurement volume within at least a part of a patient's body, providing a reference sensor establishing a first coordinate system within the measurement volume, providing a movable sensor moveable within the measurement volume, placing the patient within the measurement volume, placing the reference sensor on the skin of the patient's body, providing a virtual anatomical model of at least a part of the patient's body which is within the measurement volume, the virtual anatomical model having a second coordinate system, introducing the moveable sensor into the patient's body and moving the moveable sensor within the patient's body, aligning the first coordinate system and the second coordinate system, and translating real-time positions of the moveable sensor in the first coordinate system into positions in the second coordinate system.

The virtual anatomical model covers that part of the patient's body which is of interest, e.g. the torso with parts of the legs covering the femoral artery. The measurement volume also covers said part of the patient's body. With the tracking system, the virtual anatomical model of said part of the patient's body can be aligned with the real anatomy of the patient, so that the real anatomy of the patient matches the virtual anatomical model. When introducing e.g. an intravascular blood pump through a femoral access via the femoral artery, the movement of the movable sensor can be tracked within the virtual anatomical model, which is in alignment with the real anatomy of the patient. This allows for correct placement of the intravascular blood pump without fluoroscopy. In addition, aligning the first coordinate system of the reference sensor and the second coordinate system of the virtual anatomical model allows to detect real-time positions of the movable sensor within the virtual anatomical model independently of the relative position between the patient and the emitting device.

The step of providing a virtual anatomical model of at least a part of the of the patient's body may further include selecting one of a plurality of virtual anatomical models. The virtual anatomical model to be used may be selected by the physician before introducing the movable sensor into the patient's body. The virtual anatomical model to be used may also be automatically selected by the controller based on input parameters related to the patient. In particular, the input parameters may be sex, age, body height and/or body weight.

The reference sensor may establish a local Z-axis within the first coordinate system. The reference sensor may be placed on the skin of the patient so that the local Z-axis points toward the apex of the patient's heart. The reference sensor may comprise a cable or may have a label so that the cable or the label targets caudal. Hence, the local Z-axis may be clearly defined.

The step of aligning the first coordinate system and the second coordinate system may further comprise detecting at least a first position of the moveable sensor within the patient's body, and aligning the first coordinate system and the second coordinate system based on the detected first position. Preferably, the detected first position is an average of a plurality of spaced positions of the movable sensor, preferably of five consecutive positions. As the physician introduces the movable sensor together with e.g. the intravascular blood pump e.g. through a femoral access and pushes the same to the aorta, the actual position of the movable sensor (e.g. within the aorta) is known and can be used to align the first coordinate system and the second coordinate system.

The step of aligning the first coordinate system and the second coordinate system may further comprise detecting a movement direction of the moveable sensor within the patient's body, and aligning the first coordinate system and the second coordinate system based on the detected first position and the movement direction of the moveable sensor.

The movement direction may be detected by computing a vector between the detected first position and a detected second position, wherein the second position is detected after the first position and wherein the second position is remote from the first position. The movement direction may be calculated from a vector connecting the detected first position and a detected second position, wherein the detected second position is detected after the detected first position, i.e. when the movable sensor has been further introduced into the patient's body. Preferably, the detected second position is an average of a plurality of spaced positions, preferably of five consecutive positions. The detected first position and the detected second position are preferably sufficiently distant from each other, in particular by at least 2 cm, but no more than 10 cm. Hence, considering the movement direction further ameliorates the alignment of the first coordinate system and the second coordinate system.

The virtual anatomical model may comprise an identifiable structure and the step of aligning the first coordinate system and the second coordinate system may further comprise moving the identifiable structure together with the second coordinate system to the detected first position. The identifiable structure may be a model of an aortic arch. The identifiable structure preferably is a consistent structure independent of anatomical differences and properties between individual patients. Using an identifiable structure common over all patients further ameliorates the alignment between the first coordinate system and the second coordinate system.

The moveable sensor may be introduced into the patient's body via the femoral artery or the axillary artery.

The method of tracking the moveable sensor may further comprise the step of displaying the real-time position of the movable sensor within the provided virtual anatomical model on a display device. The method of tracking the moveable sensor may further comprise the step of displaying the real-time position of the movable sensor within the first coordinate system. This visualizes the real-time positions of the movable sensor to the physician within the virtual anatomical model which is aligned with the anatomy of the patient's body. In other words, an image of the patient's body with the real-time position of the movable sensor is shown to the physician.

### BRIEF DESCRIPTION OF THE DRAWINGS

The foregoing summary, as well as the following detailed description of exemplary embodiments, will be better understood when read in conjunction with the appended drawings. For the purpose of illustrating the present disclosure, reference is made to the drawings. The scope of the disclosure is not limited, however, to the specific embodiments disclosed in the drawings.

In the drawings:

FIG. 1 is a functional block diagram of an exemplary tracking system, in accordance with aspects of the disclosure,

FIG. 2 depicts a schematic view of the patient's body with a placed reference sensor,

FIG. 3 depicts a schematic view of a virtual anatomical model prior to alignment,

FIG. 4 depicts a schematic view of a first step of the alignment,

FIG. 5 depicts a schematic view of a second step of the alignment,

FIG. 6 depicts a schematic view of a third step of the alignment,

FIG. 7 depicts a schematic view of a fourth step of the alignment,

FIG. 8 depicts a schematic view of a fifth step of the alignment, and

FIG. 9 depicts a schematic view of the virtual anatomical model after the alignment.

### DETAILED DESCRIPTION

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

To provide an overall understanding of the systems, methods, and devices described herein, certain illustrative examples will be described. Although various examples may describe intravascular blood pumps, it will be understood that the improvements of the present technology may also be adapted and applied to other types of medical devices such as electrophysiology study and catheter ablation devices, angioplasty and stenting devices, angiographic catheters, peripherally inserted central catheters, central venous catheters, midline catheters, peripheral catheters, inferior vena cava filters, abdominal aortic aneurysm therapy devices, thrombectomy devices, TAVR delivery systems, cardiac therapy and cardiac assist devices, including balloon pumps, cardiac assist devices implanted using a surgical incision, and any other venous or arterial based introduced catheters and devices.

As is known, intravascular blood pumps can be introduced into a patient, either surgically or percutaneously, to deliver blood from one location in the heart or circulatory system to another location in the heart or circulatory system. For example, when deployed in the left ventricle, an intravascular blood pump can pump blood from the left ventricle of the heart into the aorta. When deployed in the right ventricle, an intravascular blood pump can pump blood from the inferior vena cava into the pulmonary artery.

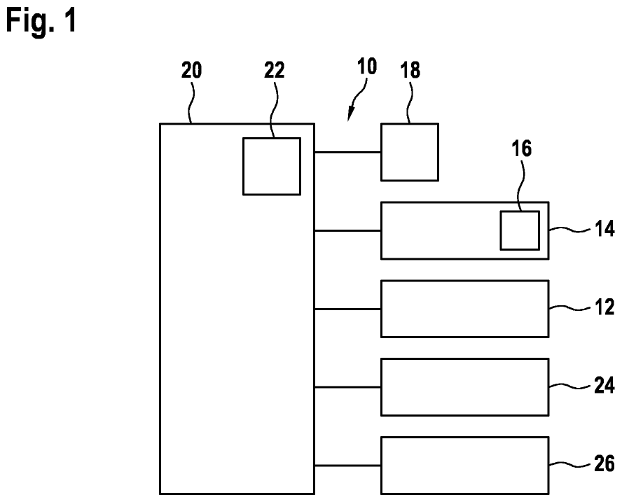

In the example of FIG. 1, a tracking system 10 comprises an emitting device 12, a medical device 14, a movable sensor 16, a reference sensor 18, a controller 20, a storage device 22, a display device 24 and an input device 26. The emitting device 12 establishes a measurement volume within a part of a patient's body B as will be described below in more detail. The emitting device 12 may be an electromagnetic field generator emitting a low-intensity, varying electromagnetic field inducing small currents in the movable sensor 16 and the reference sensor 18.

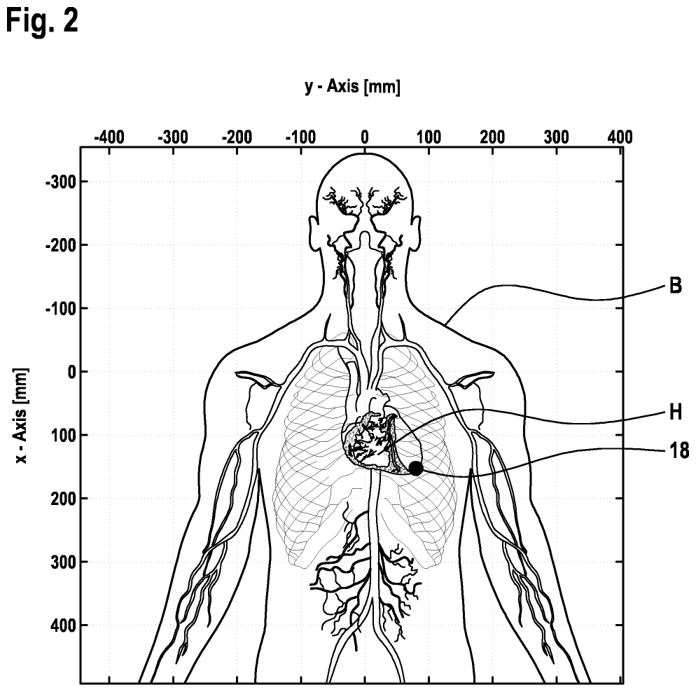

In this example, the movable sensor 16 is attached to a medical device 14, in particular to an intravascular blood pump. Here, the intravascular blood pump 14 is configured to be introduced into the patient's body B via the femoral artery in a known manner. The movable sensor 16 is an embedded electromagnetic sensor and may be a five degrees of freedom sensor or a six degrees of freedom sensor. The reference sensor 18 is a six degrees of freedom sensor and is placed on the skin of the patient's body B, as shown in FIG. 2. In particular, the reference sensor 18 establishes a first coordinate system COS1 with a local X-axis, a local Y-axis and a local Z-axis, in the following referred to as x, y and z. The reference sensor 18 is placed on the skin of the patient's body B so that the local Z-axis z of the first coordinate system COS1 points towards the apex of the patient's heart H, see FIG. 2.

The emitting device 12, the movable sensor 16 and the reference sensor 18 are connected to the controller 20 by suitable elements, e.g. by cables. Of course, it is also possible to use wireless connections. The currents induced in the movable sensor 16 and the reference sensor 18 are transmitted to the controller 20. The controller 20 is configured to amplify and digitalize the currents received. The so generated signals are further computed in the controller 20, as will be elaborated in more detail below.

The controller 20 further comprises the storage device 22. The storage device 22 may be a non-volatile storage device. In this example, a plurality of virtual anatomical models VM of at least a part of the patient's body B are stored in the storage device 22. In particular, the plurality of virtual anatomical models VM each cover an area of interest of a human body. In this example, the area of interest spans from the femoral access via the aorta AO and the aortic arch AA to the heart H. A suitable virtual anatomical model VM may be selected by the physician or may be selected automatically by the controller 22 based on input parameters like, but not limited to sex, age, height and/or weight. Each of the plurality of virtual anatomical models VM has a second coordinate system COS2 with a local X′-axis, a local Y′-axis and a local Z′-axis, in the following referred to as x′, y′ and z′.

The virtual anatomical model VM, the first coordinate system COS1, the second coordinate system COS2 and the real-time position of the movable sensor 16 in the virtual anatomical model VM, in the first coordinate system COS1 and/or the second coordinate system COS2 may be visualized to the physician via the display device 24. The display device 24 may be any suitable device, e.g. a LCD-display or a TFT-display. The physician may input different parameters and operate the tracking system 10 via the input device 26 in a known manner. The input device 26 may be any suitable device, e.g. a tablet, a keyboard, a smartphone, a remote control or a terminal. Of course, the display device 24 may comprise a touchscreen and hence, may also comprise the input device 26.

The controller 20 is configured to operate the tracking system 10 and is further configured to align the first coordinate system COS1 and the second coordinate system COS2, so that real-time positions of the movable sensor 16 in the first coordinate system COS1 are translated into positions in the second coordinate system CO2. Further, the controller 20 is configured to communicate with the display device 24 so that the virtual anatomical model VM with the position of the movable sensor 16 within the virtual anatomical model VM and the second coordinate system COS2 respectively are visualized to the physician.

Alternatively, the reference sensor 18 may also be placed on the solar plexus of the patient's body B and a further sensor may be placed on the skin of the patient's body so that the local Z-axis of the further sensor points towards the apex of the patient's heart H. This further aids the physician when introducing the blood pump 14 and facilitates the alignment of the first coordinate system COS1 and the second coordinate system COS2.

Next, the method implemented by the controller 20 to align the first coordinate system COS1 and the second coordinate system COS2 will be described in more detail with reference to FIGS. 2 to 9.

A prerequisite is that the patient's body B is placed in vicinity to the emitting device 12 so that the measurement volume covers the area of interest of the patient's body B. In the example, the area of interest spans from the femoral access to the heart H covering the aorta AO and the aortic arch AA. Further, the reference sensor 18 is attached to the patient's skin so that the local Z-axis z of the second coordinate system COS2 established by the reference sensor 18 points towards the apex of the heart H, see FIG. 2. Further, a suitable virtual anatomical model VM is selected.

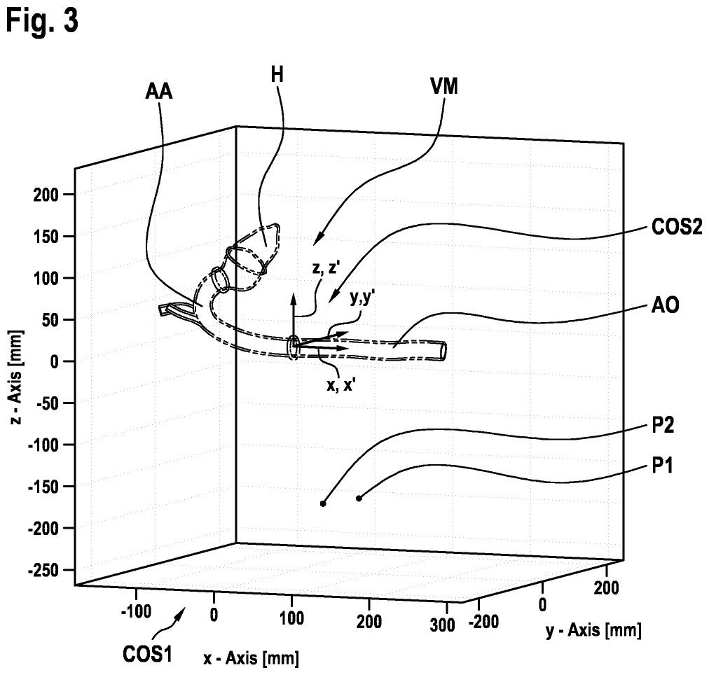

The virtual anatomical model VM is initially positioned in the origin of the reference sensor so that the first coordinate system COS1 matches the second coordinate system COS2, i.e. x=x′, y=y′ and z=z′, see FIG. 3. As shown in FIGS. 3 to 9, the virtual anatomical model comprises an identifiable structure, namely the aortic arch AO. The aortic arch AO is used as a reference structure during alignment of the second coordinate system COS2 of the virtual anatomical model with the real anatomy of the patient.

The physician introduces the intravascular blood pump 14 with the thereto attached movable sensor 16 via the femoral access and pushes the intravascular blood pump 14 cranial, towards the heart H or in reverse direction of the blood flow respectively through the descending aorta of the patient's body B. While doing so, the physician detects a detected first position P1 and a detected second position P2 in the descending aorta of the patient's body B. The detected first position P1 and the detected second position P2 each are averages of preferably five consecutive real-time positions of the movable sensor 16. The distance between the detected first position P1 and the detected second position P2 must be sufficiently high, in particular in the range of 2 cm to 10 cm. Of course, the detected first position P1 and the detected second position P2 may also be automatically detected by the controller 20. Of course, the detected first position P1 and the detected second position P2 may also be based on more or less than five consecutive real-time positions of the movable sensor 16.

The detected first position P1 and the detected second position P2 create a straight line G:\vec{x} in space, in axial direction of the descending aorta. Further, the straight line G:\vec{x} also depicts a vector denoting the starting position (P1) and the movement direction of the moveable sensor 16 and hence, of the medical device 14.

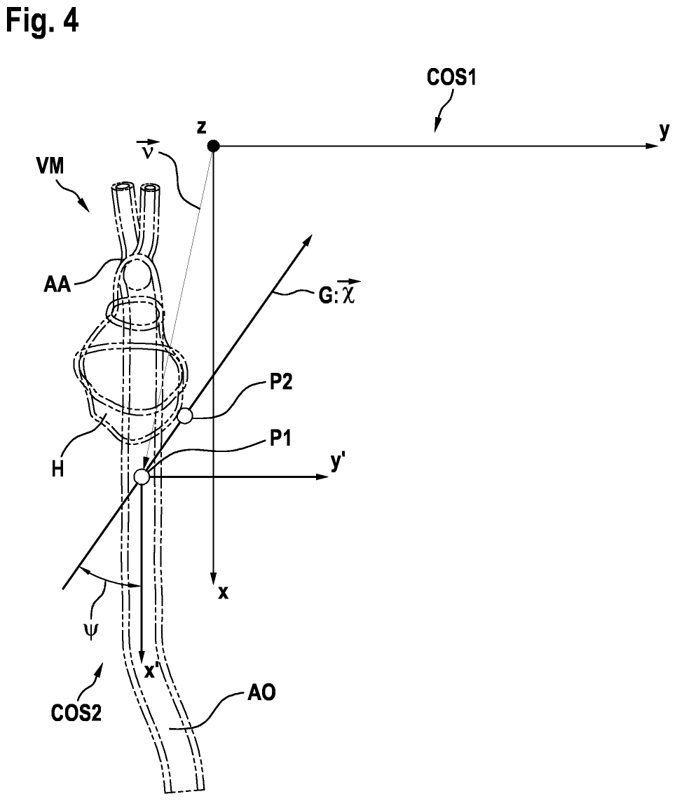

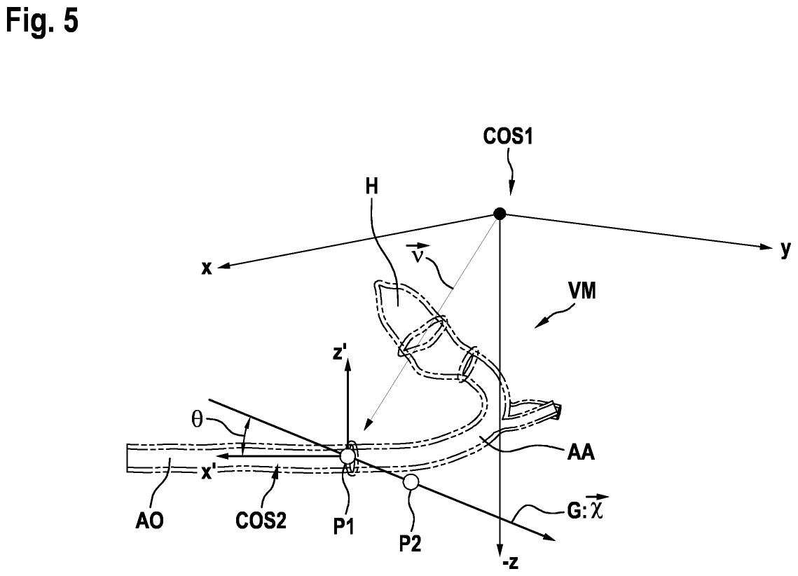

As shown in FIG. 4, the aortic arch AA of the virtual anatomical model VM is translated along a vector \vec{v} onto the detected first position P1. Next, the aortic arch AA of the virtual anatomical model VM is rotated around z′ by an angle ψ. The angle ψ is the angle between x′ and the straight line G:\vec{x}. FIG. 5 shows the virtual anatomical model VM after the rotation around z′.

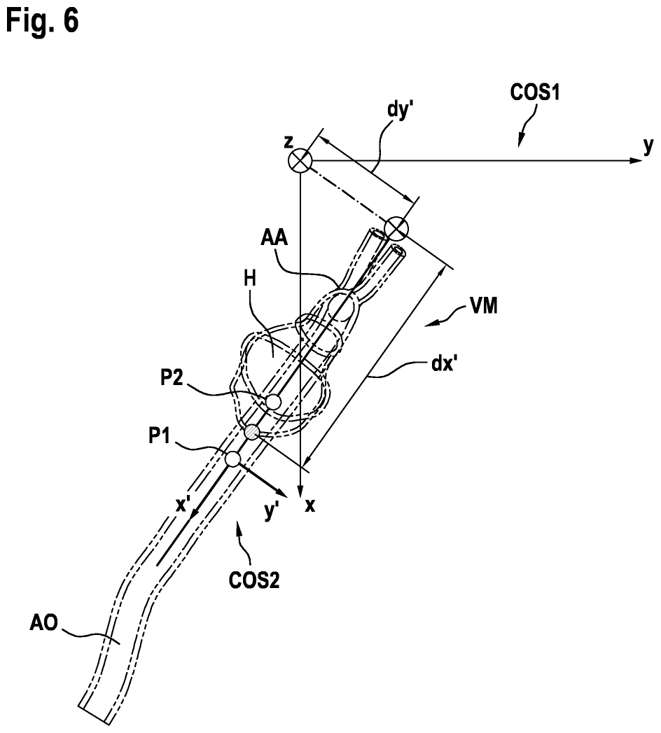

Next, the aortic arch AA of the virtual anatomical model VM is rotated around y′ by angle θ, which is in reference to the x-y-plane, see FIG. 5. The angle θ spans between the straight line G:\vec{x} and x′. As a result of this second rotation, x′ of the aortic arch AA of the virtual anatomical model VM is now aligned to the straight line G:\vec{x}. FIG. 6 shows the aortic arch AA of the virtual anatomical model VM after the second rotation. As can be seen, x′ (which is axially concentric with the descending aorta) is aligned with the straight line G:\vec{x}.

The aortic arch AA of the virtual anatomical model VM is then shifted along the straight line G:\vec{x} or x′ respectively up to the shortest distance between the apex of the heart H and z (i.e. the Z-axis of the first coordinate system COS1 established by the reference sensor 18). Therefore, the straight line G:\vec{x} is parallel shifted to pass through the apex of the aortic arch AA of the virtual anatomical model VM. Further, the apex of the heart H of the virtual anatomical model VM is then rotated around the x′ axis resulting in a shift of dy′ in y′-direction and in x′-direction by dx′, as shown in FIG. 6. The shortest distance between the straight line G:\vec{x} and z is calculated in a known manner based on the distance of skew lines.

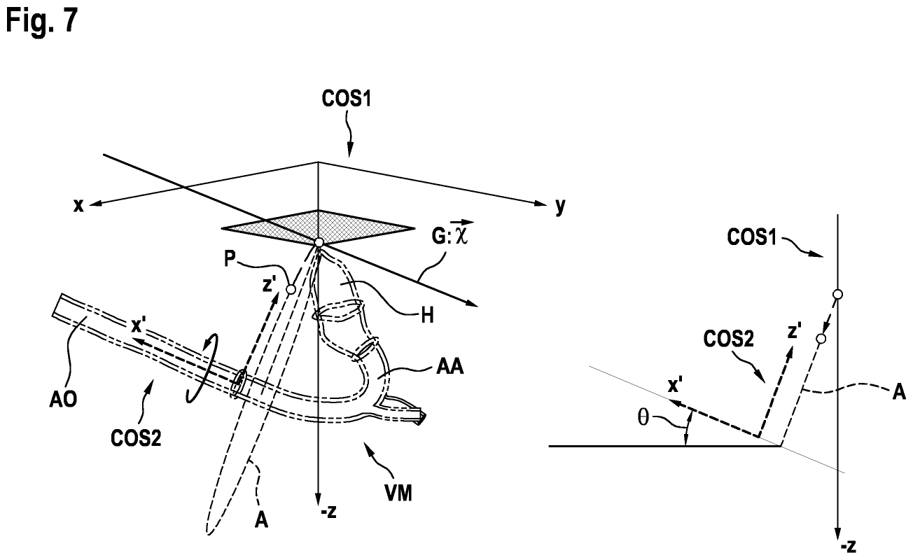

To adjust the aortic arch AA of the virtual anatomical model VM with respect to a rotation around y′, the shift dx′ in x′-direction has to be corrected by dx″ under certain circumstances. As one can take from the left side of FIG. 7, a rotation of the aortic arch AA (used as a reference structure) of the virtual anatomical model VM in plane A around x′ is required to move the apex of the aortic arch AA of the virtual anatomical model VM, so that it is located on z (i.e. the Z-axis of the first coordinate system COS1). The right side of FIG. 7 shows a view perpendicular to plane A in y′-direction. In case x′ is parallel to the x-y-plane, no additional adjustment is required, as θ=0°. I.e. in case dy′=0, there is no need for rotation around x′ and no additional adjustment is required. However, in case the aortic arch AA of the virtual anatomical model VM is generically located within the first coordinate system COS1, i.e. θ≠0°, a rotation of the aortic arch AA of the virtual anatomical model VM around x′ would not result in the apex of the aortic arch AA of the virtual anatomical model VM lying on z.

Only in the latter case (i.e. θ≠0°) the shift in x′-direction dx′ has to be adjusted by dx″ as follows:

$${dx}^{″} = tan θ * Δ R = tan θ * ( 1 - cos ( φ ) ) * R$$

$$with$$

$$sin φ = \frac{{dy}^{′}}{R} \to φ = arc sin ( \frac{{dy}^{′}}{R} ) .$$

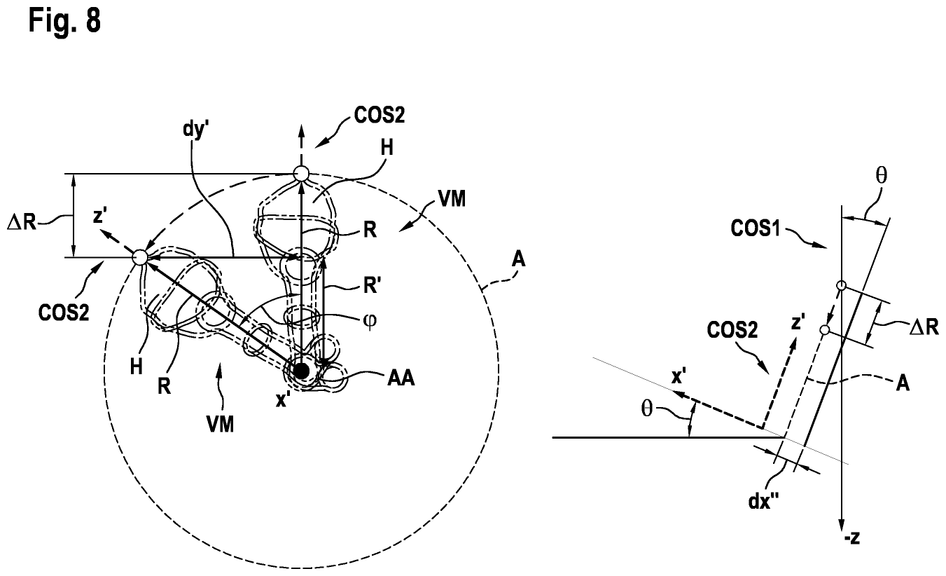

The angle φ depicts the angle the aortic arch AA of the virtual anatomical model has to be rotated around x′ to move the apex of the aortic arch AA of the virtual anatomical model VM to z, see FIG. 8. Here, the left side of FIG. 8 shows a front view onto the plane A and the right side of FIG. 8 shows a perpendicular view to the plane A. As one can further take from FIG. 8, the distance R is the distance between x′ and the apex of the aortic arch AA of the virtual anatomical model VM. Accordingly, the distance R′ is the difference between R and ΔR, with ΔR depicting the absolute difference in z′-direction due to the rotation around x′ by angle, wherein

$$cos φ = \frac{{R}^{′}}{R} \to cos φ * R = R - Δ R$$

$$and$$

$$Δ R = ( 1 - cos φ ) * R .$$

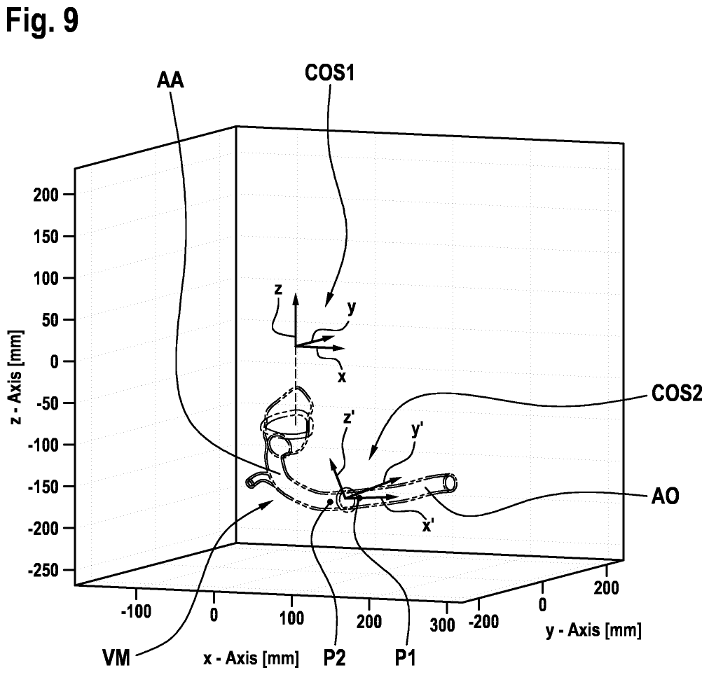

Now, the first coordinate system COS1 and the second coordinate system COS2 are aligned so that the virtual anatomical model VM matches the real anatomic properties of the patient, as shown in FIG. 9. The controller 20 transmits the respective signals to the display device 24 so that the virtual anatomical model VM with the position of the movable sensor 16 within the virtual anatomical model VM and the second coordinate system COS2 respectively is visualized to the physician, allowing for a correct placement of the medical device 14 in the patient's heart H.

### Exemplary Implementations

As already described, the technology described herein may be implemented in various ways. In that regard, the foregoing disclosure is intended to include, but not be limited to, the systems, methods, and combinations and subcombinations thereof that are set forth in the following exemplary implementations. Preferred embodiments are described in the following paragraphs:
A1 Tracking system comprising:
   an emitting device configured to establish a measurement volume within at least a part of a patient's body,
   a moveable sensor which is moveable within the measurement volume,
   a reference sensor establishing a first coordinate system within the measurement volume,
   a storage device with at least one virtual anatomical model of at least a part of the patient's body, wherein the at least one virtual anatomical model has a second coordinate system, and a controller being configured to align the first coordinate system and the second coordinate system and to translate real-time positions of the moveable sensor in the first coordinate system into positions in the second coordinate system.
A2 Tracking system according paragraph A1, wherein the controller is configured to detect real-time positions of the movable sensor within the first coordinate system.
A3 Tracking system according to paragraph A1 or A2, wherein the controller is configured to align the first coordinate system and the second coordinate system based on at least a detected first position of the moveable sensor within the first coordinate system.
A4 Tracking system according to paragraph A3, wherein the controller is configured to align the first coordinate system and the second coordinate system based on the detected first position and a movement direction of the movable sensor, wherein the movement direction is preferably a linear vector.
A5 Tracking system according to paragraph A3 or A4, wherein the virtual anatomical model comprises an identifiable structure and wherein the controller is further configured to move the identifiable structure together with the second coordinate system to the detected first position.
A6 Tracking system according to any one of the preceding paragraphs A1 to A5, wherein the virtual anatomical model comprises a model of the at least one blood vessel, in particular of an aortic arch, a femoral artery and/or an aorta.
A7 Tracking system according to any one of the preceding paragraphs A1 to A6, wherein the reference sensor is a six degrees of freedom sensor.
A8 Tracking system according to any one of the preceding paragraphs A1 to A7, further comprising a medical device, wherein the moveable sensor is attached to the medial device, and wherein the medical device is preferably an intravascular blood pump or a guidewire for an intravascular blood pump.
A9 Tracking system according to any one the preceding paragraphs A1 to A8, wherein the emitting device is an electromagnetic field generator.
A10 Tracking system according to any one of the preceding paragraphs A1 to A9, wherein the moveable sensor is an embedded electromagnetic sensor and/or wherein the movable sensor is a five degrees of freedom sensor or a six degrees of freedom sensor.
A11 Tracking system according to any one of the preceding paragraphs A1 to A10, further comprising a display device configured to display the real-time positions of the moveable sensor in the second coordinate system of the virtual anatomical model.
A12 Tracking system according to paragraph A11, wherein the display device is connected to the controller.
A13 Tracking system according to any one of the preceding paragraphs A1 to A12, wherein the storage device comprises a plurality of selectable virtual anatomical models.
A14 Tracking system according to any one of the preceding paragraphs A1 to A13, further comprising an input device configured to communicate with the controller.
A15 Tracking system according to paragraph A14, wherein the input device is a touch screen, a keyboard, a cell phone, a wireless input device, a wired input device, a terminal, a tablet, and/or a remote control.
A16 Tracking system according to any one of the preceding paragraphs A1 to A15, wherein the controller comprises the storage device.
A17 Tracking system according to any one of the preceding paragraphs A1 to A16, wherein the storage device is a non-volatile storage device.
A18 Method of tracking real-time positions a moveable sensor within a patient's body with a tracking system, in particular with a tracking system according to one of the preceding paragraphs A1 to A17, the method comprising the following steps:
   Providing an emitting device establishing a measurement volume within at least a part of a patient's body,
   Providing a reference sensor establishing a first coordinate system within the measurement volume,
   Providing a movable sensor moveably within the measurement volume,
   placing the patient within the measurement volume,
   placing the reference sensor on the skin of the patient's body,
   providing a virtual anatomical model of at least a part of the patient's body which is within the measurement volume, the virtual anatomical model having a second coordinate system,
   introducing the moveable sensor into the patient's body and moving the moveable sensor within the patient's body,
   aligning the first coordinate system and the second coordinate system, and
   translating real-time positions of the moveable sensor in the first coordinate system into positions in the second coordinate system.
A19 Method according to paragraph A18, wherein providing a virtual anatomical model of at least a part of the patient's body includes selecting one of a plurality of virtual anatomical models.
A20 Method according to paragraph A18 or A19, wherein the reference sensor establishes a local Z-axis within the first coordinate system, and wherein the reference sensor is placed on the skin of the patient so that the local Z-axis points towards the apex of the patient's heart.
A21 Method according to any one of the preceding paragraphs A18 to A20, further comprising the step of:
   tracking the movement of the movable sensor by aligning the first coordinate system and the second coordinate system, and by translating real-time positions of the moveable sensor in the first coordinate system into positions in the second coordinate system.
A22 Method according to any one of the preceding paragraphs A18 to A21, wherein aligning the first coordinate system and the second coordinate system further comprises:
   detecting at least a first position of the moveable sensor within the patient's body, and
   aligning the first coordinate system and the second coordinate system based on the detected first position.
A23 Method according to any one the preceding paragraphs A18 to A22, wherein aligning the first coordinate system and the second coordinate system further comprises:
   detecting a movement direction of the moveable sensor within the patient's body, and
   aligning the first coordinate system and the second coordinate system based on the detected first position and the movement direction of the moveable sensor.
A24 Method according to paragraph A22 or A23, wherein the virtual anatomical model comprises an identifiable structure and wherein aligning the first coordinate system and the second coordinate system further comprises:
   moving the identifiable structure together with the second coordinate system to the detected first position.
A25 Method according to paragraph A24, wherein the identifiable structure is a model of an aortic arch.
A26 Method according to any one of the preceding paragraphs A18 to A25, wherein the moveable sensor is introduced into the patient's body via the femoral artery or via the axillary artery.
A27 Method according to any one of the preceding paragraphs A18 to A26, further comprising the step of:
   displaying the real-time position of the movable sensor within the provided virtual anatomical model on a display device.
A28 Method according to any one of the preceding paragraphs A18 to A27, further comprising the step of:
   displaying the real-time position of the movable sensor within the second coordinate system on a display device.

### LIST OF REFERENCE SIGNS

10 tracking system
12 emitting device
14 medical device/intravascular blood pump
16 moveable sensor
18 reference sensor
20 controller
22 storage device
24 display device
26 input device
A plane
AA aortic arch
AO aorta
B patient's body
COS1 first coordinate system
COS2 second coordinate system
dx′ shift in x′ direction
dx″ correction of dx′
dy′ shift in y′ direction
G:\vec{x} straight line between P1 and P2
H heart
P1 detected first position
P2 detected second position
R distance between x′ and apex of aortic arch of virtual anatomical model
R′ distance
ΔR difference between R and R′
\vec{v} vector
VM virtual anatomical model
X local X-axis of first coordinate system
y local Y-axis of first coordinate system
Z local Z-axis of first coordinate system
x′ local X-axis of second coordinate system
y′ local Y-axis of second coordinate system
local Z-axis of second coordinate system
ψ angle between x′ and G:\vec{x}

1. A tracking system comprising:
an emitting device configured to establish a measurement volume within at least a part of a patient's body;
a moveable sensor which is moveable within the measurement volume;
a reference sensor establishing a first coordinate system within the measurement volume;
a storage device comprising at least one virtual anatomical model of at least a part of the patient's body, wherein the at least one virtual anatomical model has a second coordinate system; and
a controller configured to align the first coordinate system and the second coordinate system and to translate real-time positions of the moveable sensor in the first coordinate system into positions in the second coordinate system.

2. The tracking system according to claim 1, wherein the controller is configured to detect real-time positions of the moveable sensor within the first coordinate system.

3. The tracking system according to claim 1, wherein the controller is configured to align the first coordinate system and the second coordinate system based on at least a detected first position of the moveable sensor within the first coordinate system.

4. The tracking system according to claim 3, wherein the controller is configured to align the first coordinate system and the second coordinate system based on the detected first position and a movement direction of the moveable sensor, wherein the movement direction is a linear vector.

5. The tracking system according to claim 3, wherein the at least one virtual anatomical model comprises an identifiable structure and wherein the controller is further configured to move the identifiable structure together with the second coordinate system to the detected first position.

6. The tracking system according to claim 1, wherein the at least one virtual anatomical model comprises a model of at least one blood vessel, wherein the at least one blood vessel comprises an aortic arch-(AA), a femoral artery and/or an aorta.

7. The tracking system according to claim 1, wherein the reference sensor is a six degrees of freedom sensor.

8. The tracking system according to claim 1, further comprising a medical device, wherein the moveable sensor is attached to the medical device, and wherein the medical device comprises an intravascular blood pump or a guidewire for an intravascular blood pump.

9. The tracking system according to claim 1, wherein the emitting device is an electromagnetic field generator.

10. The tracking system according to claim 1, wherein the moveable sensor is an embedded electromagnetic sensor and/or wherein the moveable sensor is a five degrees of freedom sensor or a six degrees of freedom sensor.

11. The tracking system according to claim 1, further comprising a display device configured to display the real-time positions of the moveable sensor in the second coordinate system of the at least one virtual anatomical model.

12. The tracking system according to claim 1, wherein the storage device comprises a plurality of selectable virtual anatomical models.

13. The tracking system according to claim 1, further comprising an input device configured to communicate with the controller.

14. A method of tracking real-time positions of a moveable sensor within a patient's body with a tracking system, the method comprising:
providing an emitting device establishing a measurement volume within at least a part of a body of a patient;
providing a reference sensor establishing a first coordinate system within the measurement volume;
providing a moveable sensor moveably within the measurement volume,
placing the patient within the measurement volume;
placing the reference sensor on skin of the body of the patient;
providing a virtual anatomical model of at least a part of the body of the patient which is within the measurement volume, the virtual anatomical model having a second coordinate system;
introducing the moveable sensor into the body of the patient and moving the moveable sensor within the body of the patient;
aligning the first coordinate system and the second coordinate system; and
translating real-time positions of the moveable sensor in the first coordinate system into positions in the second coordinate system.
